(19) United States
(12) Patent Application Publication
(10) Pub. No.: US 2014/0114581 A1
(43) Pub. Date: Apr. 24, 2014

(54) USING GAME THEORY IN IDENTIFYING COMPOUNDS THAT BIND TO TARGETS

(71) Applicants: Christopher J. Langmead, Monroeville, PA (US); Hetunandan Kamisetty, Seattle, WA (US)

(72) Inventors: Christopher J. Langmead, Monroeville, PA (US); Hetunandan Kamisetty, Seattle, WA (US)

(73) Assignee: CARNEGIE MELLON UNIVERSITY, Pittsburgh, PA (US)

(21) Appl. No.: 14/001,932

(22) PCT Filed: Feb. 28, 2012
(86) PCT No.: PCT/US2012/026966

§ 371 Date: Dec. 13, 2013

Related U.S. Application Data
(60) Provisional application No. 61/464,149, filed on Feb. 28, 2011.

(51) Int. Cl. G01N 33/566

(52) U.S. Cl. CPC G01N 33/566
USPC 702/19

(57) ABSTRACT

A method performed by one or more processing devices includes retrieving information indicative of compounds and mutations of a target; generating one or more predictions of binding affinities of the compounds to the mutations; generating a simulation for introducing at least one of the compounds in response to at least one of the mutations; identifying, based on an application of a game theory algorithm to the simulation and to the one or more predictions, an equilibrium of the simulation; and identifying, from the equilibrium, a subset of the compounds with an increased binding affinity to the mutations relative to other binding affinities of other of the compounds.

### CLAIM OF PRIORITY

This application claims priority to provisional U.S. Patent Application 61/464,149, filed on Feb. 28, 2011, the entire contents of which are hereby incorporated by reference.

### GOVERNMENT RIGHTS

The techniques disclosed herein are made with government support under the National Science Foundation, grant number IIS-0905193. The government may have certain rights in the techniques disclosed herein.

### BACKGROUND

When a drug is given to a patient for treatment of a virus, the virus often responds by acquiring mutations that confer resistance to the drug. The phenomenon of drug resistance is costly in terms of lives and money.

### SUMMARY

In one aspect of the present disclosure, a method performed by one or more processing devices includes retrieving information indicative of compounds and mutations of a target; generating one or more predictions of binding affinities of the compounds to the mutations; generating a simulation for introducing at least one of the compounds in response to at least one of the mutations; identifying, based on an application of a game theory algorithm to the simulation and to the one or more predictions, an equilibrium of the simulation; and identifying, from the equilibrium, a subset of the compounds with an increased binding affinity to the mutations relative to other binding affinities of other of the compounds.

Implementations of the disclosure can include one or more of the following features. In some implementations, the method also includes retrieving information indicative of compounds and mutations of a target; generating one or more predictions of binding affinities of the compounds to the mutations; generating a simulation for introducing at least one of the compounds in response to at least one of the mutations; identifying, based on an application of a game theory algorithm to the simulation and to the one or more predictions, an equilibrium of the simulation; and identifying, from the equilibrium, a subset of the compounds with an increased binding affinity to the mutations relative to other binding affinities of other of the compounds.

In other implementations, the method also includes identifying, from the equilibrium, a distribution of probabilities that the mutations arise in response to the compounds. In still other implementations, the simulation includes a game; the game comprises a target player and a compound player; the target player corresponds to a target; the compound player corresponds to an entity that generates compounds; the target player is associated with a first plurality of actions to be performed during execution of the game; at least one of the first plurality of actions includes introducing a mutation into the game; the compound player is associated with a second plurality of actions to be formed during execution of the game; at least one of the second plurality of actions includes introducing a compound into the game; execution of the game generates a utility for a compound-target pair resulting from at least one of the first plurality of actions and at least one of the second plurality of actions; and a utility is indicative of a binding affinity.

In some implementations, a compound includes one or more of a drug, a combination of drugs, a nucleic acid, and a polymer; and a target includes one or more of a protein, an enzyme, and a nucleic acid. In still other implementations, the equilibrium includes a Correlated Equilibrium.

In some implementations, the subset includes a cocktail, and the method further includes: identifying information indicative of a percentage of each compound in the subset to be included in the cocktail. In other implementations, generating the one or more predictions includes: applying a regression model to (i) properties of the compounds retrieved, and (ii) properties of the mutations retrieved; wherein the regression model is associated with relationships among properties of known compounds, properties of known mutations, and binding affinities among the known compound and the known mutations.

In still another aspect of the disclosure, one or more machine-readable media are configured to store instructions that are executable by one or more processing devices to perform operations including retrieving information indicative of compounds and mutations of a target; generating one or more predictions of binding affinities of the compounds to the mutations; generating a simulation for introducing at least one of the compounds in response to at least one of the mutations; identifying, based on an application of a game theory algorithm to the simulation and to the one or more predictions, an equilibrium of the simulation; and identifying, from the equilibrium, a subset of the compounds with an increased binding affinity to the mutations relative to other binding affinities of other of the compounds. Implementations of this aspect of the present disclosure can include one or more of the foregoing features.

In still another aspect of the disclosure, an electronic system includes one or more processing devices; and one or more machine-readable media configured to store instructions that are executable by the one or more processing devices to perform operations including: retrieving information indicative of compounds and mutations of a target; generating one or more predictions of binding affinities of the compounds to the mutations; generating a simulation for introducing at least one of the compounds in response to at least one of the mutations; identifying, based on an application of a game theory algorithm to the simulation and to the one or more predictions, an equilibrium of the simulation; and identifying, from the equilibrium, a subset of the compounds with an increased binding affinity to the mutations relative to other binding affinities of other of the compounds. Implementations of this aspect of the present disclosure can include one or more of the foregoing features.

All or part of the foregoing can be implemented as a computer program product including instructions that are stored on one or more non-transitory machine-readable storage media, and that are executable on one or more processing devices. All or part of the foregoing can be implemented as an apparatus, method, or electronic system that can include one or more processing devices and memory to store executable instructions to implement the stated functions.

The details of one or more implementations are set forth in the accompanying drawings and the description below. Other features, objects, and advantages will be apparent from the description and drawings, and from the claims.

### BRIEF DESCRIPTION OF DRAWINGS

FIG. 1 is a diagram of an example of a network environment for identifying compounds that bind to targets.

FIG. 2 is a block diagram showing examples of components of a network environment for identifying compounds that bind to targets.

FIG. 3 is a flowchart showing an example process for identifying compounds that bind to targets.

FIG. 4 shows an example of a computer device and a mobile computer device that can be used to implement the techniques described herein.

Like reference symbols and designations in the various drawings indicate like elements.

### DETAILED DESCRIPTION

A system consistent with this disclosure implements a game theory technique for identifying compounds that bind to targets. The system may also be used to predict how the target will evolve (e.g., which mutations will arise) in response to a fixed compound (or a fixed cocktail of such compounds). Generally, a target includes an item for which an effect is measured. Types of targets include proteins, enzymes, nucleic acids, and so forth. Generally, a compound includes a material. Types of compounds include drugs, combinations of drugs (e.g., drug cocktails), chemicals, polymers, nucleic acids and so forth.

Game theory includes a study of strategic scenarios between players in a game (e.g., a particular situation). In game theory, a utility of a player's actions depends on the actions made by the other players. One of the primary interests in game theory is computing equilibrium strategies for specific games. An equilibrium strategy is a joint probability distribution over actions where no player has an incentive to deviate from the distribution.

In an example, the system is configured to compute various equilibrium strategies, including, e.g., a Nash Equilibrium, a Correlated Equilibrium, and so forth. Generally, a Nash Equilibrium includes a strategy that factorizes over a set of players. Generally, a Correlated Equilibrium generalizes the Nash Equilibrium by allowing for arbitrary joint distributions over actions.

Some diseases are associated with evolution of a target through mutations. These mutations can decrease or abolish the efficacy of a compound, leading to a phenomenon known as resistance. By identifying a compound that binds to mutated targets, the system is configured to identify compounds with increased effectiveness against mutations, e.g., relative to effectiveness of other compounds against the mutations.

To address the problem of drug resistance, the system implements a game theory technique to identify a compound that binds to targets with mutations. In an example, the system is configured to generate a simulation that models interactions between a compound (and/or a plurality of compounds) and a target (and/or a plurality of targets). Generally, a simulation includes an environment for predicting outcomes of various scenarios. In an example, the simulation includes a game. The game includes players. Generally, a player includes a participant in the game. In an example, the players include a compound designer and a target. Generally, a compound designer includes an entity that designs compounds.

During the game, the target and the compound designer each make moves. Generally, a move includes performance of an action during the game. The target makes moves by introducing mutations. In this example, the target selects mutations that increase disruption of a compound binding to the target, e.g., to emulate drug resistance. The compound designer makes moves by selecting one of several compounds. The compound designer selects compounds that are predicted to promote binding of the compound to a target, e.g., in response to the mutation.

A move is associated with a utility value (hereinafter utility). Generally, a utility includes an amount of value that a player derives from a move. In an example, a utility of a set of moves includes a binding affinity of a compound to a mutated target.

In an example, a set of moves result in a target-compound pair that has binding affinity above a threshold value. In this example, the system determines that the compound designer wins the game (e.g., the compound designer has designed a compound that binds to the target with the mutations).

In another example, the set of moves result in a target-compound pair that has binding affinity below the threshold value. In this example, the system determines that the compound designer loses the game (e.g., the compound designer has designed a compound that cannot bind to the target with the mutations). The aim of the compound designer is to find a compound (e.g., a drug or a drug cocktail) that binds well against moves of the target.

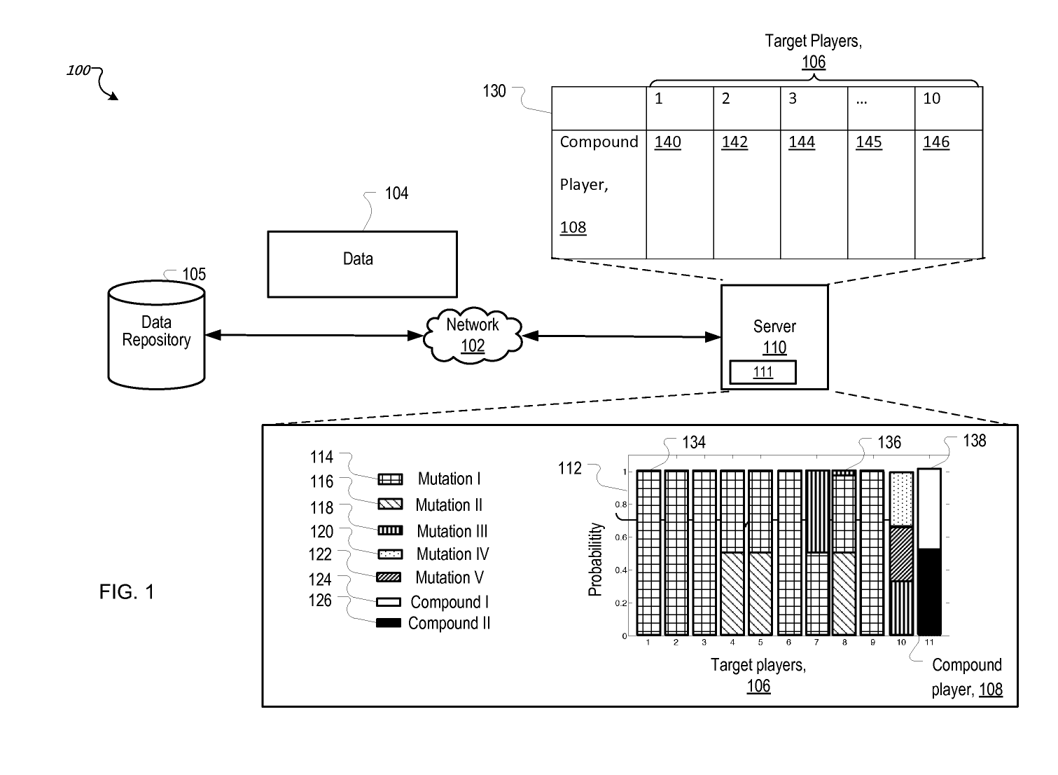

FIG. 1 is a diagram of an example of network environment 100 for identifying compounds 124, 126 that bind to targets (and/or to mutations 114, 116, 118, 120, 122 of a target). Network environment 100 includes network 102, data repository 105, and server 110.

Data repository 105 can communicate with server 110 over network 102. Network environment 100 may include many thousands of data repositories and servers, which are not shown. Server 110 may include various data engines, including, e.g., data engine 111. Although data engine 111 is shown as a single component in FIG. 1, data engine 111 can exist in one or more components, which can be distributed and coupled by network 102.

In the example of FIG. 1, data engine 111 retrieves, from data repository 105, data 104. In this example, data 104 includes data indicative of a target and data indicative of one or more compounds.

In an example, a target includes a sequence of amino acids. In this example, there are twenty-two different types of amino acids. The sequence includes a plurality of positions. Each position can be populated with one of the twenty-two different amino acids.

A target includes a default sequence of amino acids, in which various positions are populated with various types of amino acids. In this example, a mutation of the target includes a change in a type of at least one of the amino acids in at least one of the positions as compared to the unmutated target sequence.

In the example of FIG. 1, data 104 includes data indicative of various mutations for a target. In an example, some mutations of a target are non-functional (e.g., not energetically viable). In this example, data 104 includes mutations that are functional for a target (e.g., mutations that are energetically viable). In another example, data 104 includes all possible mutations, functional or otherwise. In this example, data engine 111 is configured to identify mutations that are functional and resistant. Data 104 also includes information indicative of one or more compounds. In this example, the compounds include compounds that have been generated by a compound designer (not shown). In a variation of FIG. 1, the information indicative of one or more compounds may be received by server 110 from a computing device used the compound designer.

Using data 104, data engine 111 generates game 130. Game 130 includes target players 106 and compound player 108. Generally, a target player includes a player representing a position in the sequence of a target. In the example of FIG. 1, data 104 indicates that the target includes at least ten positions in the sequence. In this example, data engine 111 generates target players 106 for each of these ten positions. Generally, a compound player includes a player representing a compound designer.

In the example of FIG. 1, target players 106 may make various moves, e.g., during execution of game 130. Data 104 indicates that there are five functional mutations for the positions of the target. The five mutations are visually represented as mutations 114, 116, 118, 120, 122. In game 130, target players 106 may make moves by introducing one of mutations 114, 116, 118, 120, 122 into game 130.

Compound player 108 also makes moves in game 130. In an example, compound player 130 makes moves by choosing a compound to be applied to one or more mutations 114, 116, 118, 120, 122. In the example of FIG. 1, data 104 indicates that there are two different compounds that may be used by compound player 108. These compounds are visually represented by compounds 124, 126.

In the example of FIG. 1, one of target players 106 makes a move by introducing one or more mutations 114, 116, 118, 120, 122 into game 130. In response, compound player 106 introduces one of compounds 124, 126 into game 130. During each iteration of game play, data engine 111 generates a utility for the moves of the target players 106 and the compound player 108. In another example, data engine 111 computes the utilities without simulating each iteration of game play. In this example, data engine 111 is configured to automatically generate the utilities.

In an example, a binding affinity of a compound (e.g., introduced by compound player 108) to a mutated target (e.g., introduced by one of target players 106) is indicative of a utility generated by the various moves. Generally, a binding affinity includes a value indicative of a strength of bond between a target (e.g., an amino acid in a target) and a compound. In an example, data engine 111 implements a regression model (e.g., a ridge regression model, a least squares regression model, and so forth) in predicting utilities, e.g., a binding affinity of various compounds to mutations. In another example, a utility includes a value indicative of an effectiveness of a compound binding to a mutated target.

For example, game 130 may include portions 140, 142, 144, 145, 146. In this example, data engine 111 is configured to populate portions 140, 142, 144, 145, 146 with values indicative of utilities associated with various moves of target players 106 and compound player 108. Data engine 111 uses the utilities in computing an equilibrium of game 130.

In an example, data engine 111 implements a game theory technique to compute an equilibrium for game 130. In this example, the game theory technique is configured to use a utility function for each of target players 106 and compound player 106. Generally, a utility function includes a set of utilities that are associated with various moves that are available to players in a game. In this example, data engine 111 uses the utilities that are computed using the above-described techniques in generating utility functions for target players 106 and compound player 106.

In an example, the game theory technique includes execution of various rules and/or algorithms for calculating a Nash Equilibrium. In another example, the game theory technique includes execution of various rules and/or algorithms for calculating a Correlated Equilibrium.

In the example of FIG. 1, data engine 111 generates visual representation 112 of a Correlated Equilibrium for target players 106 and compound players 108 in game 130. Visual representation 112 includes an x-axis and a y-axis. The y-axis includes probability values ranging from a value of zero to a value of one. The x-axis includes values indicative of target players 106 and compound player 108.

Visual representation 112 includes various distributions associated with target players 106 and compound player 108. In this example, the distributions are represented in columns, e.g., columns 134, 136, 138. The columns associated with target players 106 include data indicative of an optimal strategy for target players 106. Column 138 associated with compound player 108 includes data indicative of an optimal strategy for compound player 108.

In the example of FIG. 1, column 134 indicates an optimal strategy for a target player associated with a first position in an amino acid sequence of the target. In this example, the optimal strategy includes mutating the first position in accordance with mutation 114 in response to all compounds that are applied to the target.

Column 136 indicates an optimal strategy for a target player associated with an eighth position in an amino acid sequence of the target. In this example, column 136 is indicative of a set of mutations 114, 116, 118 that are likely to arise in response to compounds 124, 126. In this example, the optimal strategy includes mutating the eighth position in accordance with (i) mutation 118 ten percent of the time, (ii) mutation 114 forty-five percent of the time, and (iii) mutation 116 forty-five percent of the time. An optimal strategy for the target player associated with the eighth position is to generate (i) mutation 118 ten percent of the time, (ii) mutation 114 forty-five percent of the time, and (iii) mutation 116 forty-five percent of the time.

Column 138 includes data specifying a compound that increases a binding affinity for mutations 114, 116, 118, 120, 122, e.g., relative to other binding affinities for other compounds and mutations 114, 116, 118, 120, 122. In this example, column 138 indicates that the compound is a combination of compounds 124, 126. Column 138 also indicates the composition of the compound, namely, that the compound includes fifty percent of compound 124 and fifty percent of compound 126. In this example, column 138 includes information indicative of composition of a compound that exhibits an increased binding affinity across a combination of mutations 114, 116, 118, 120, 122, e.g., relative to other binding affinities across the combination of mutations 114, 116, 118, 120, 122.

In an example, data engine 111 determines a winner of game 130. Generally, a winner includes a player of game 130 that is associated with a utility value above or below a threshold value. If the utility value is above the threshold value, one player wins. If the utility value is below the threshold value, another player wins.

In an example, game 130 results in an equilibrium with a utility value below the threshold value. In this example, a utility value below the threshold value corresponding to an expected binding energy that is negative and therefore favorable. In this example, compound player 108 wins game 130. In another example, game 130 results in an equilibrium with a utility value above the threshold value. In this example, the target wins game 130.

In a variation of FIG. 1, a game may include multiple compound players. In this example, an equilibrium of the game identifies a combinatorial set of possible compounds that increases a binding affinity for mutations introduced into the game, e.g., relative to other binding affinities for other cocktails and the mutations introduced into the game.

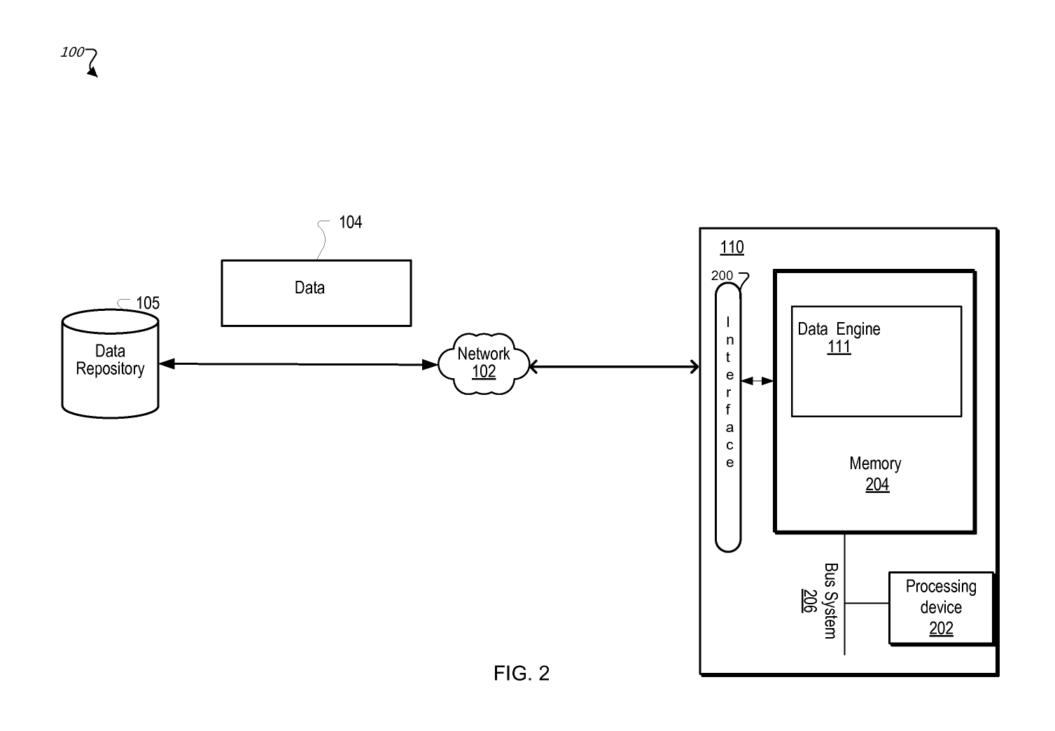

FIG. 2 is a block diagram showing examples of components of network environment 100 for identifying compounds 124, 126 that bind to targets (and/or to mutations 114, 116, 118, 120, 122 of a target). In the example of FIG. 2, visual representation 112, mutations 114, 116, 118, 120, 122, compounds 124, 126 and game 130 are not shown.

Network 102 can include a large computer network, including, e.g., a local area network (LAN), wide area network (WAN), the Internet, a cellular network, or a combination thereof connecting a number of mobile computing devices, fixed computing devices, and server systems. The network(s) may provide for communications under various modes or protocols, including, e.g., Transmission Control Protocol/Internet Protocol (TCP/IP), Global System for Mobile communication (GSM) voice calls, Short Message Service (SMS), Enhanced Messaging Service (EMS), or Multimedia Messaging Service (MMS) messaging, Code Division Multiple Access (CDMA), Time Division Multiple Access (TDMA), Personal Digital Cellular (PDC), Wideband Code Division Multiple Access (WCDMA), CDMA2000, or General Packet Radio System (GPRS), among others. Communication may occur through a radio-frequency transceiver. In addition, short-range communication may occur, including, e.g., using a Bluetooth, WiFi, or other such transceiver.

Server 110 can be a variety of computing devices capable of receiving data and running one or more services, which can be accessed by data repository 105. In an example, server 110 can include a server, a distributed computing system, a desktop computer, a laptop, a cell phone, a rack-mounted server, and the like. Server 110 can be a single server or a group of servers that are at a same location or at different locations. Data repository 105 and server 110 can run programs having a client-server relationship to each other. Although distinct modules are shown in the figures, in some examples, client and server programs can run on the same device.

Server 110 can receive data from data repository 105 through input/output (I/O) interface 200. I/O interface 200 can be a type of interface capable of receiving data over a network, including, e.g., an Ethernet interface, a wireless networking interface, a fiber-optic networking interface, a modem, and the like. Server 110 also includes a processing device 202 and memory 204. A bus system 206, including, for example, a data bus and a motherboard, can be used to establish and to control data communication between the components of server 110.

Processing device 202 can include one or more microprocessors. Generally, processing device 202 can include an appropriate processor and/or logic that is capable of receiving and storing data, and of communicating over a network (not shown). Memory 204 can include a hard drive and a random access memory storage device, including, e.g., a dynamic random access memory, or other types of non-transitory machine-readable storage devices. As shown in FIG. 2, memory 204 stores computer programs that are executable by processing device 202. These computer programs include data engine 111. Data engine 111 can be implemented in software running on a computer device (e.g., server 110), hardware or a combination of software and hardware.

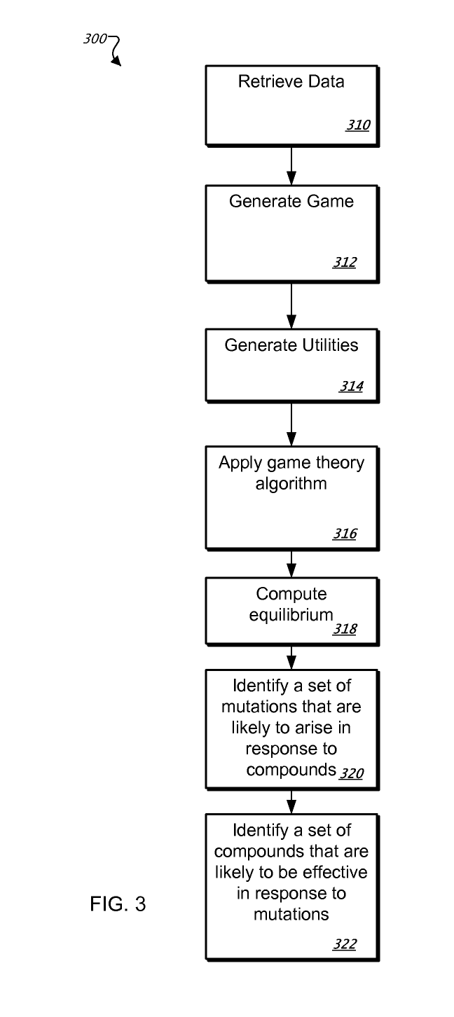

FIG. 3 is a flowchart showing an example process 300 for identifying compounds 124, 126 that bind to targets (and/or to mutations 114, 116, 118, 120, 122 of a target). In FIG. 3, process 300 is performed on server 110 (and/or by data engine 111 on server 110).

In operation, data engine 111 retrieves (310) data 104 from data repository 105. In an example, data 104 is indicative of mutations 114, 116, 118, 120, 122 that are functional for a target (not shown). Data 104 also includes information indicative of a number of positions in the sequence of amino acids for the target. As described herein, data engine 111 generates a number of target players 106 in accordance with the number of positions and/or in accordance with a subset of the number of positions.

Data 104 also includes data indicative of compounds 124, 126. Using data 104, data engine 111 generates (312) game 130. In this example, game 130 includes target players 106 and compound player 108. As previously described, each of target players 106 corresponds to a position in a sequence (e.g., an amino acid sequence) of a target. Target players 106 may perform one or more moves by introducing one or more of mutations 114, 116, 118, 120, 122 into game 130.

Compound player 108 corresponds to a compound designer. As previously described, compound player 108 may perform one or more moves in game 130 by introducing one or more of compounds 124, 126 in response to one or more of mutations 114, 116, 118, 120, 122.

Data engine 111 generates (314) utilities for the various combinations of mutations 114, 116, 118, 120, 122 and compounds 124, 126. In an example, data engine 111 generates predictions of utilities using a linear regression model that predicts the binding effect of a compound on a mutated target. In an example, the linear regression model is associated with relationships among properties of compounds and properties of mutations.

In this example, data engine 111 accesses, from data repository 105, information indicative of properties of mutations 114, 116, 118, 120, 122, properties of compounds 124, 126, results of prior experiments of binding various compounds to the various mutated targets (e.g., binding affinities), and properties of the mutated targets and compounds in the experiments. Data engine 111 generates the predictions based on a comparison of (i) the properties of one or more of mutations 114, 116, 118, 120, 122 and properties of one or more of compounds 124, 126, to (ii) the properties of the mutated targets and compounds in the experiments that resulted in binding affinities with increased values, e.g., relative to other binding affinities resulting from the experiments.

Using the predicted utilities, data engine 111 applies (316) a game theory algorithm to game 130. In an example, the game theory algorithm includes an algorithm for computing a Nash Equilibrium. In another example, the game theory algorithm includes an algorithm for computing a Correlated Equilibrium.

Based on application of the game theory algorithm, data engine 111 computes (318) an equilibrium for game 130. In an example, data engine 111 generates visual representation 112 of the equilibrium. In an example, the equilibrium includes a distribution over compounds 124, 126 and a distribution over mutations 114, 116, 118, 120, 122.

From the equilibrium, data engine 111 identifies (320) a set of mutations that are likely to arise in response to compounds 124, 126. For example, from column 136, data engine 111 identifies a set of mutations 114, 116, 118 that are likely to arise in an eighth position in an amino acid sequence in the target, e.g., in response to compounds 124, 126.

Data engine 111 also identifies (322) a set of compounds 124, 126 that are likely to be effective in response to mutations 114, 116, 118, 120, 122. In the example of FIG. 1, column 138 includes information indicative of percentages of compounds 124, 126 to be included in a cocktail, e.g., to promote the cocktail having an increased binding affinity to target mutations.

Using the techniques described herein, a system implements a game theory technique for identifying compounds that bind to targets. From implementation of the game theory technique, the system identifies an optimal distribution of compounds in cocktail, e.g., to promote binding of the cocktail to mutated targets.

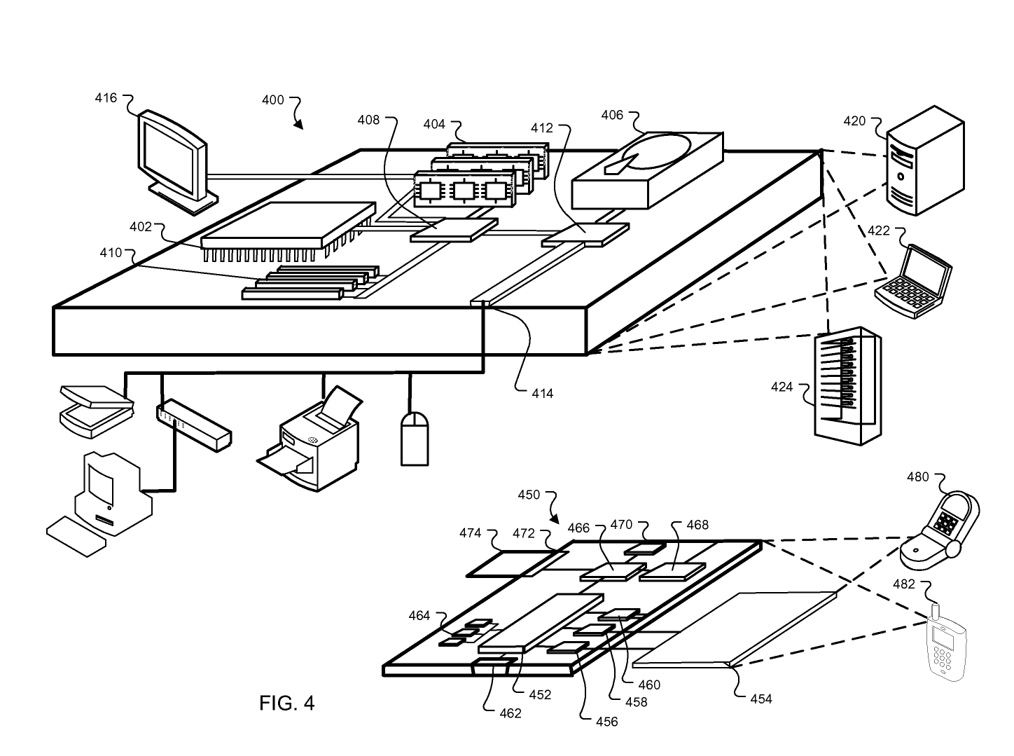

FIG. 4 shows an example of computer device 400 and mobile computer device 450, which can be used with the techniques described here. Computing device 400 is intended to represent various forms of digital computers, such as laptops, desktops, workstations, personal digital assistants, servers, blade servers, mainframes, and other appropriate computers. Computing device 450 is intended to represent various forms of mobile devices, such as personal digital assistants, cellular telephones, smartphones, and other similar computing devices. The components shown here, their connections and relationships, and their functions, are meant to be examples only, and are not meant to limit implementations of the techniques described and/or claimed in this document.

Computing device 400 includes processor 402, memory 404, storage device 406, high-speed interface 408 connecting to memory 404 and high-speed expansion ports 410, and low speed interface 412 connecting to low speed bus 414 and storage device 406. Each of components 402, 404, 406, 408, 410, and 412, are interconnected using various busses, and can be mounted on a common motherboard or in other manners as appropriate. Processor 402 can process instructions for execution within computing device 400, including instructions stored in memory 404 or on storage device 406 to display graphical data for a GUI on an external input/output device, such as display 416 coupled to high speed interface 408. In other implementations, multiple processors and/or multiple buses can be used, as appropriate, along with multiple memories and types of memory. Also, multiple computing devices 400 can be connected, with each device providing portions of the necessary operations (e.g., as a server bank, a group of blade servers, or a multi-processor system).

Memory 404 stores data within computing device 400. In one implementation, memory 404 is a volatile memory unit or units. In another implementation, memory 404 is a non-volatile memory unit or units. Memory 404 also can be another form of computer-readable medium, such as a magnetic or optical disk.

Storage device 406 is capable of providing mass storage for computing device 400. In one implementation, storage device 406 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in a data carrier. The computer program product also can contain instructions that, when executed, perform one or more methods, such as those described above. The data carrier is a computer- or machine-readable medium, such as memory 404, storage device 406, memory on processor 402, and the like.

High-speed controller 408 manages bandwidth-intensive operations for computing device 400, while low speed controller 412 manages lower bandwidth-intensive operations. Such allocation of functions is an example only. In one implementation, high-speed controller 408 is coupled to memory 404, display 416 (e.g., through a graphics processor or accelerator), and to high-speed expansion ports 410, which can accept various expansion cards (not shown). In the implementation, low-speed controller 412 is coupled to storage device 406 and low-speed expansion port 414. The low-speed expansion port, which can include various communication ports (e.g., USB, Bluetooth®, Ethernet, wireless Ethernet), can be coupled to one or more input/output devices, such as a keyboard, a pointing device, a scanner, or a networking device such as a switch or router, e.g., through a network adapter.

Computing device 400 can be implemented in a number of different forms, as shown in the figure. For example, it can be implemented as standard server 420, or multiple times in a group of such servers. It also can be implemented as part of rack server system 424. In addition or as an alternative, it can be implemented in a personal computer such as laptop computer 422. In some examples, components from computing device 400 can be combined with other components in a mobile device (not shown), such as device 450. Each of such devices can contain one or more of computing device 400, 450, and an entire system can be made up of multiple computing devices 400, 450 communicating with each other.

Computing device 450 includes processor 452, memory 464, an input/output device such as display 454, communication interface 466, and transceiver 468, among other components. Device 450 also can be provided with a storage device, such as a microdrive or other device, to provide additional storage. Each of components 450, 452, 464, 454, 466, and 468, are interconnected using various buses, and several of the components can be mounted on a common motherboard or in other manners as appropriate.

Processor 452 can execute instructions within computing device 450, including instructions stored in memory 464. The processor can be implemented as a chipset of chips that include separate and multiple analog and digital processors. The processor can provide, for example, for coordination of the other components of device 450, such as control of user interfaces, applications run by device 450, and wireless communication by device 450.

Processor 452 can communicate with a user through control interface 458 and display interface 456 coupled to display 454. Display 454 can be, for example, a TFT LCD (Thin-Film-Transistor Liquid Crystal Display) or an OLED (Organic Light Emitting Diode) display, or other appropriate display technology. Display interface 456 can include appropriate circuitry for driving display 454 to present graphical and other data to a user. Control interface 458 can receive commands from a user and convert them for submission to processor 452. In addition, external interface 462 can communicate with processor 442, so as to enable near area communication of device 450 with other devices. External interface 462 can provide, for example, for wired communication in some implementations, or for wireless communication in other implementations, and multiple interfaces also can be used.

Memory 464 stores data within computing device 450. Memory 464 can be implemented as one or more of a computer-readable medium or media, a volatile memory unit or units, or a non-volatile memory unit or units. Expansion memory 474 also can be provided and connected to device 450 through expansion interface 472, which can include, for example, a SIMM (Single In Line Memory Module) card interface. Such expansion memory 474 can provide extra storage space for device 450, or also can store applications or other data for device 450. Specifically, expansion memory 474 can include instructions to carry out or supplement the processes described above, and can include secure data also. Thus, for example, expansion memory 474 can be provide as a security module for device 450, and can be programmed with instructions that permit secure use of device 450. In addition, secure applications can be provided via the SIMM cards, along with additional data, such as placing identifying data on the SIMM card in a non-hackable manner.

The memory can include, for example, flash memory and/or NVRAM memory, as discussed below. In one implementation, a computer program product is tangibly embodied in a data carrier. The computer program product contains instructions that, when executed, perform one or more methods, such as those described above. The data carrier is a computer- or machine-readable medium, such as memory 464, expansion memory 474, and/or memory on processor 452, that can be received, for example, over transceiver 468 or external interface 462.

Device 450 can communicate wirelessly through communication interface 466, which can include digital signal processing circuitry where necessary. Communication interface 466 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through radio-frequency transceiver 468. In addition, short-range communication can occur, such as using a Bluetooth®, WiFi, or other such transceiver (not shown). In addition, GPS (Global Positioning System) receiver module 470 can provide additional navigation- and location-related wireless data to device 450, which can be used as appropriate by applications running on device 450.

Device 450 also can communicate audibly using audio codec 460, which can receive spoken data from a user and convert it to usable digital data. Audio codec 460 can likewise generate audible sound for a user, such as through a speaker, e.g., in a handset of device 450. Such sound can include sound from voice telephone calls, can include recorded sound (e.g., voice messages, music files, and the like) and also can include sound generated by applications operating on device 450.

Computing device 450 can be implemented in a number of different forms, as shown in the figure. For example, it can be implemented as cellular telephone 480. It also can be implemented as part of smartphone 482, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions.

To provide for interaction with a user, the systems and techniques described here can be implemented on a computer having a display device (e.g., a CRT (cathode ray tube) or LCD (liquid crystal display) monitor) for displaying data to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, the engines described herein can be separated, combined or incorporated into a single or combined engine. The engines depicted in the figures are not intended to limit the systems described here to the software architectures shown in the figures.

A number of embodiments have been described. Nevertheless, it will be understood that various modifications can be made without departing from the spirit and scope of the processes and techniques described herein. In addition, the logic flows depicted in the figures do not require the particular order shown, or sequential order, to achieve desirable results. In addition, other steps can be provided, or steps can be eliminated, from the described flows, and other components can be added to, or removed from, the described systems. Accordingly, other embodiments are within the scope of the following claims.

What is claimed is:

1. A method performed by one or more processing devices, comprising:
retrieving information indicative of compounds and mutations of a target;
generating one or more predictions of binding affinities of the compounds to the mutations;
generating a simulation for introducing at least one of the compounds in response to at least one of the mutations;
identifying, based on an application of a game theory algorithm to the simulation and to the one or more predictions, an equilibrium of the simulation; and
identifying, from the equilibrium, a subset of the compounds with an increased binding affinity to the mutations relative to other binding affinities of other of the compounds.

2. The method of claim 1, further comprising:
identifying, from the equilibrium, a distribution of probabilities that the mutations arise in response to the compounds.

3. The method of claim 1,
wherein the simulation comprises a game;
wherein the game comprises a target player and a compound player;
wherein the target player corresponds to a target;
wherein the compound player corresponds to an entity that generates compounds;
wherein the target player is associated with a first plurality of actions to be performed during execution of the game;
wherein at least one of the first plurality of actions comprises introducing a mutation into the game;
wherein the compound player is associated with a second plurality of actions to be formed during execution of the game;
wherein at least one of the second plurality of actions comprises introducing a compound into the game;
wherein execution of the game generates a utility for a compound-target pair resulting from at least one of the first plurality of actions and at least one of the second plurality of actions; and
wherein a utility is indicative of a binding affinity.

4. The method of claim 1,
wherein a compound comprises one or more of a drug, a combination of drugs, a nucleic acid, and a polymer; and
wherein a target comprises one or more of a protein, an enzyme, and a nucleic acid.

5. The method of claim 1, wherein the equilibrium comprises a Correlated Equilibrium.

6. The method of claim 1, wherein the subset comprises a cocktail, and wherein the method further comprises:
identifying information indicative of a percentage of each compound in the subset to be included in the cocktail.

7. The method of claim 1, wherein generating the one or more predictions comprises:
applying a regression model to (i) properties of the compounds retrieved, and (ii) properties of the mutations retrieved;
wherein the regression model is associated with relationships among properties of known compounds, properties of known mutations, and binding affinities among the known compound and the known mutations.

8. One or more machine-readable media configured to store instructions that are executable by one or more processing devices to perform operations comprising:
retrieving information indicative of compounds and mutations of a target;
generating one or more predictions of binding affinities of the compounds to the mutations;
generating a simulation for introducing at least one of the compounds in response to at least one of the mutations;
identifying, based on an application of a game theory algorithm to the simulation and to the one or more predictions, an equilibrium of the simulation; and
identifying, from the equilibrium, a subset of the compounds with an increased binding affinity to the mutations relative to other binding affinities of other of the compounds.

9. The one or more machine-readable media of claim 8, wherein the operations further comprise:
identifying, from the equilibrium, a distribution of probabilities that the mutations arise in response to the compounds.

10. The one or more machine-readable media of claim 8,
wherein the simulation comprises a game;
wherein the game comprises a target player and a compound player;
wherein the target player corresponds to a target;
wherein the compound player corresponds to an entity that generates compounds;
wherein the target player is associated with a first plurality of actions to be performed during execution of the game;
wherein at least one of the first plurality of actions comprises introducing a mutation into the game;
wherein the compound player is associated with a second plurality of actions to be formed during execution of the game;
wherein at least one of the second plurality of actions comprises introducing a compound into the game;
wherein execution of the game generates a utility for a compound-target pair resulting from at least one of the first plurality of actions and at least one of the second plurality of actions; and
wherein a utility is indicative of a binding affinity.

11. The one or more machine-readable media of claim 8,
wherein a compound comprises one or more of a drug, a combination of drugs, a nucleic acid, and a polymer; and
wherein a target comprises one or more of a protein, an enzyme, and a nucleic acid.

12. The one or more machine-readable media of claim 8, wherein the equilibrium comprises a Correlated Equilibrium.

13. The one or more machine-readable media of claim 8, wherein the subset comprises a cocktail, and wherein the operations further comprise:
identifying information indicative of a percentage of each compound in the subset to be included in the cocktail.

14. The one or more machine-readable media of claim 8, wherein generating the one or more predictions comprises:
applying a regression model to (i) properties of the compounds retrieved, and (ii) properties of the mutations retrieved;
wherein the regression model is associated with relationships among properties of known compounds, properties of known mutations, and binding affinities among the known compound and the known mutations.

15. An electronic system comprising:
one or more processing devices; and
one or more machine-readable media configured to store instructions that are executable by the one or more processing devices to perform operations comprising:
retrieving information indicative of compounds and mutations of a target;
generating one or more predictions of binding affinities of the compounds to the mutations;
generating a simulation for introducing at least one of the compounds in response to at least one of the mutations;
identifying, based on an application of a game theory algorithm to the simulation and to the one or more predictions, an equilibrium of the simulation; and
identifying, from the equilibrium, a subset of the compounds with an increased binding affinity to the mutations relative to other binding affinities of other of the compounds.

16. The electronic system of claim 15, wherein the operations further comprise:
identifying, from the equilibrium, a distribution of probabilities that the mutations arise in response to the compounds.

17. The electronic system of claim 15,
wherein the simulation comprises a game;
wherein the game comprises a target player and a compound player;
wherein the target player corresponds to a target;
wherein the compound player corresponds to an entity that generates compounds;
wherein the target player is associated with a first plurality of actions to be performed during execution of the game;
wherein at least one of the first plurality of actions comprises introducing a mutation into the game;
wherein the compound player is associated with a second plurality of actions to be formed during execution of the game;
wherein at least one of the second plurality of actions comprises introducing a compound into the game;
wherein execution of the game generates a utility for a compound-target pair resulting from at least one of the first plurality of actions and at least one of the second plurality of actions; and
wherein a utility is indicative of a binding affinity.

18. The electronic system of claim 15,
wherein a compound comprises one or more of a drug, a combination of drugs, a nucleic acid, and a polymer; and
wherein a target comprises one or more of a protein, an enzyme, and a nucleic acid.

19. The electronic system of claim 15, wherein the equilibrium comprises a Correlated Equilibrium.

20. The electronic system of claim 15, wherein the subset comprises a cocktail, and wherein the operations further comprise:
identifying information indicative of a percentage of each compound in the subset to be included in the cocktail.

21. The electronic system of claim 15, wherein generating the one or more predictions comprises:
applying a regression model to (i) properties of the compounds retrieved, and (ii) properties of the mutations retrieved;
wherein the regression model is associated with relationships among properties of known compounds, properties of known mutations, and binding affinities among the known compound and the known mutations.
